(19) United States
(12) Patent Application Publication
(10) Pub. No.: US 2008/0312577 A1
(43) Pub. Date: Dec. 18, 2008

(54) EXPANDABLE DIALYSIS APPARATUS AND METHOD

(75) Inventors: William J. Drasler, Minnetonka, MN (US); Mark L. Jenson, Greenfield, MN (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 12/026,038

(22) Filed: Feb. 5, 2008

Related U.S. Application Data
(60) Provisional application No. 60/899,602, filed on Feb. 5, 2007.

(51) Int. Cl. A61M 5/32

(52) U.S. Cl. 604/6.16

(57) ABSTRACT

A venovenous expandable dialysis apparatus includes a blood feeding needle component configured to introduce blood at a position of a first peripheral vein and a blood withdrawal needle component configured to withdraw blood at another position from a second peripheral vein located, where the first position is located away from the second position. The expandable dialysis apparatus further includes a guide wire having a central axis, an expanding sheath configured circumferentially around the guide wire to form an annular lumen between a distal blood withdrawal position and a proximal extracorporeal position; and a needle disposed around the expandable sheath.

### CROSS-REFERENCE TO RELATED APPLICATIONS

This application claims the benefit of U.S. Provisional Application No. 60/899,602, filed Feb. 5, 2007, the contents of which are incorporated herein by reference.

### FIELD OF THE INVENTION

The invention relates to extracorporeal hemodialysis. More particularly, the invention related to a method and apparatus for dialyzing a patient's blood with a pair of peripherally inserted venipuncture or cannulation.

### BACKGROUND OF THE INVENTION

Each year, millions of people die from some type of kidney problems. Some of these problems include: infections, kidney stones, kidney cancer and Polycystic Kidney Disease. Many people also have chronic kidney disease (CKD), where the kidneys do not work as well as they should and lead to kidney failure such that the kidney ceases to remove waste and excess water from the blood. Kidney failure can only be treated with dialysis or a kidney transplant. For patients requiring a dialysis, three primary methods of dialysis exist in hemodialysis: an intravenous catheter, an arteriovenous (AV) Cimino fistula, or synthetic graft.

An intravenous catheter access, sometimes called a CVC (Central Venous Catheter), consists of a plastic catheter with two lumens (or occasionally two separate catheters) which is inserted into a large vein (usually the vena cava, via the internal jugular vein or the femoral vein) to allow large flows of blood to be withdrawal from one lumen, to go into the dialysis circuit, and to be returned via the other lumen. However blood flow is almost always less than that of a well functioning fistula or graft.

Aside from infection, venous stenosis is another serious problem with catheter access. The catheter is a foreign body in the vein, and often provokes an inflammatory reaction in the vein wall, which results in scarring and narrowing of the vein, often to the point where it occludes. This can cause problems with severe venous congestion in the area drained by the vein and may also render the vein, and the veins drained bay it, useless for the formation of a fistula or graft at a later date. Patients on longterm hemodialysis can literally ‘run-out’ of access, so this can be a fatal problem.

Difficulties in creating and maintaining vascular access in haemodialysis patients often require insertion of long-term dual-lumen central venous catheters (permcaths). One of the most common problems encountered with permcaths is poor blood flow, most often secondary to the formation of a fibrin sheath around the lumens.

Catheter access is usually used for rapid access for immediate dialysis, for tunnelled access in patients who are deemed likely to recover from acute renal failure, and patients with end-stage renal failure, who are either waiting for alternative access to mature, or those who are unable to have alternative access.

Catheter access is often popular with patients, as attachment to the dialysis machine doesn't require needles. However the serious risks of catheter access noted above mean that such access should only be contemplated as a long term solution in the most desperate access situation.

The most commnonly accepted practice for dialyzing a patient's blood extracorporeally requires the surgical creation of a subcutaneous, arterio-venous fistula. Thereafter, the subcutaneous venous system dilates secondary to the increase of blood flow derived from the artery to the vein through the fistula. Sufficient blood flow for dialysis is then obtainable by venipuncture with large bore needles. Normally, two hollow needles or cannulas are used to perform two venipunctures on the patient so that two blood-communication sites exist simultaneously in the patient. Conventionally, blood is withdrawn through one needle, forced through a hemodialyzer and thereafter forced through the other module. The needles have to be substantially distant from one another to prevent recirculation of blood.

The aforementioned procedure has been found to have serious disadvantages both to the patient and to the attending physicians, nurses, and technicians. The problems are particularly aggravated because most patients requiring extracorporeal hemodialysis must undergo treatment as frequently as three to four times per week. This means that if every venipuncture were completely successful, a patient would need to undergo from 6 to 8 venipunctures or cannulations each week.

It is well-known that the duration and well-function of a fistula created by venipuncture is inversely related to the number of venipunctures. Tissue repeatedly subjected to the trauma of venipuncture is much more susceptible to paravascular hemorrhage, clotting and infection. In fact, it is commonly found in patients who have experienced a number of venipunctures, that the tissues surrounding the most accessible veins develop large hematomas which obscure the veins, making successful venipuncture extremely difficult because of insufficient blood flow in the damaged blood vessels.

Existing apparatus and method for blood access and for dialysis are not optimal. Another problem is the large caliber of access devices, such as large diameter dialysis needles, which are more painful, more traumatic, and which produce more delayed hemostasis after needle removal than would smaller caliber devices; however, the large caliber is required with the present devices in order to obtain the required flow rate through the devices with acceptable pressures.

Another problem is the shear forces and resulting damage to the natural and synthetic vessels and vascular grafts and adjacent tissues. One other shortcoming is that standard apparatus and methods are not practical for use in home or more routine clinical settings, but requires specialized dialysis facilities with capacity for treating urgent bleeding complications which can occur. Yet another shortcoming is the permanent arteriovenous shunting which is required for standard chronic hemodialysis access methods. The present invention addresses problems and shortcomings of the existing apparatus for blood access and for hemodialysis.

### SUMMARY OF THE INVENTION

The invention is an expendable sheath and related apparatus, particularly adapted for vascular access such as for hemodialysis. More particularly, the apparatus allows smaller and less traumatic punctures of the vascular wall (native vessel or prosthetic graft or intravascular graft) while providing a larger diameter along a substantial portion of the cannula to reduce resistance to flow of blood or other fluids. Thus, a long catheter (or sheath, cannula, etc.) may be employed, which would otherwise the impractical due to excessive flow resistance. The long catheter lengths allow the distal tip to be petitioned more centrally in the vasculature than would otherwise be practical. The longer practical length and small vessel puncture can be utilized to access the central venous vessels such as subclavian vein or vena cava from a peripheral puncture such as in the arm.

A vascular graft or stent graft adapted for dialysis access may be implanted in one or both arms to utilize as puncture sites. An elastomeric graft is particularly advantageous for this purpose, but any vascular prosthesis could be utilized.

The present invention provides for reduced shear forces on access puncture, and provides enhanced radial expansion of the puncture; these enhancements provide for reduced trauma at the puncture.

The invention also provides reduced vessel or graft compression during puncture, because of the reduced needle or cannula size compared to the standard devices and methods. One possible application for this new cannula is in veno-venous dialysis. Venovenous dialysis offers advantages in decreased bleeding over that with standard approaches, and may make lower cost or home hemodialysis approaches feasible.

Other applications include enhanced arteriovenous dialysis, improved central venous access for diagnostic or therapeutic procedures such as obtaining central blood samples and administration of chemotherapy medication. The invention includes vascular access apparatus, methods of vascular access, and methods of fabrication.

The invention is pointed out with particularity in the appended claims; however, other objects, uses, features and advantages, together with methods for malting and using the invention will be evident or become apparent from a consideration of the following detailed description of the preferred embodiments and practices included in the invention and from the drawings herewith,

### BRIEF DESCRIPTION OF THE DRAWINGS

FIG. 1 is a schematic depiction of the expandable dialysis apparatus of the present invention having a needle, an expandable sheath and a guidewire.

FIG. 2 is a schematic depiction of a portion of the expandable sheath and the guidewire of the expandable dialysis apparatus of FIG. 1.

FIG. 3 schematically depicts the use of the of the expandable dialysis apparatus of FIG. 1 in a lumen, either native or synthetics in which the needle is introduced into the lumen.

FIG. 4 schematically depicts advancing the expandable sheath and guidewire through the needle positions in FIG. 3.

FIG. 5 schematically depicts the withdrawing of the needle of FIG. 4.

FIG. 6 schematically depicts the withdrawing of the guidewire of FIG. 5.

FIG. 7 schematically depicts the expansion of the sheath of FIG. 6 with the lumen.

FIG. 8 schematically depicts the use of a supporting catheter with the sheath of FIG. 7.

FIGS. 9-11 depict alternate embodiments of the catheter of FIG. 8.

FIG. 12 depicts a side view of a puncture through a lumen wall according to the prior art.

FIG. 13 depicts a side view of a puncture through a lumen wall according to the present invention.

FIG. 14 depicts a top planar view of a puncture through a graft wall according to the prior art.

FIGS. 15-16 depict a top planar view of a puncture through a graft wall according to the present invention.

FIGS. 17-18 depict use of the expandable sheaths of the present invention for dialysis treatment.

### DETAILED DESCRIPTION OF PREFERRED EMBODIMENTS

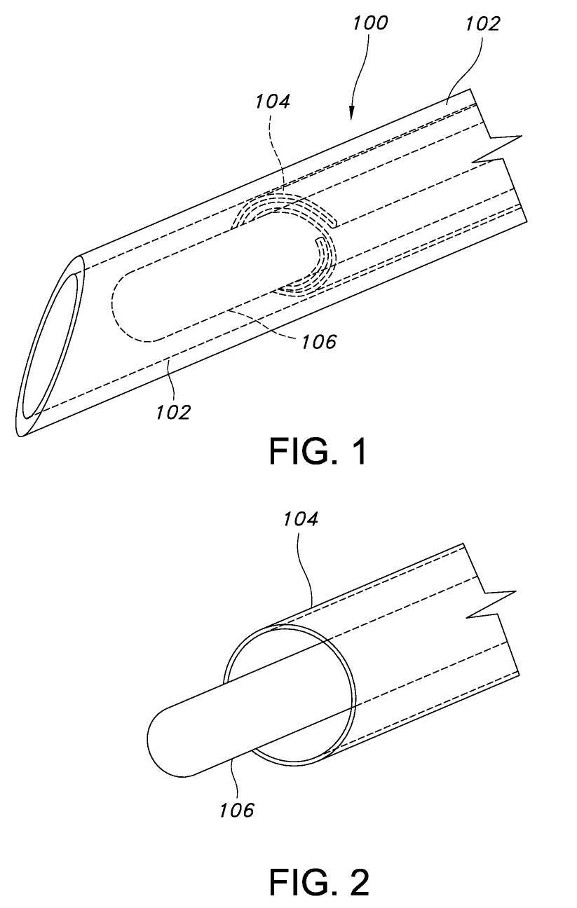

As shown, FIG. 1 sets forth an expandable cannula apparatus 100, particularly adapted to provide low flow resistance with a small vascular puncture. An important aspect of the apparatus is an expandable sheath 104 which has a small introduction profile for lesser trauma at the puncture site but which expands to a larger size inside the blood vessel. The expandable sheath 104 is introduced into the lumen through a needle 102. In addition, the expandable sheath 104 may be provided circumferentially around the guide wire 106.

For higher pressure applications, such as the infusion cannula for hemodialysis, the expandable sheath may be utilized without additional support. For low pressure applications such as the withdrawal cannula for hemodialysls, an internal supporting catheter can also be used to facilitate passage of another intravascular device by reducing frictional forces which would otherwise exist between the intravascular device and the expandable sheath.

A portion of or all of the expandable sheath 104 may be wrapped around the guide wire 106 or other catheter device, rolled into a small profile alongside the guide wire or other catheter device, passed through a lumen of a catheter device, or otherwise configured in a small profile and flexible configuration for introduction at a vascular puncture site.

Various means may be provided to temporarily retain the expandable sheath 104 in its small profile configuration, such as elastic recoil or memory, plastic deformation such as by thermally setting the configuration, retention by adhesives, fibers, loops, tubes or other devices.

As depicted in FIG. 27 after passage introduction into a vessel (not shown), the expandable sheath 104 is expanded to provide a lumen for passage of blood, saline, medication, contrast, or other material, or for passage of a supporting or treatment catheter. The action of expanding the expandable sheath 104 may be accomplished by a variety of mechanisms. The simplest expansion mechanism is simply to supply pressurized fluid such as blood or saline to the proximal end of the expandable sheath, and allow the supply pressure to expand the expandable sheath; alternatively, advancing a support catheter within the expandable sheath will expand the expandable sheath.

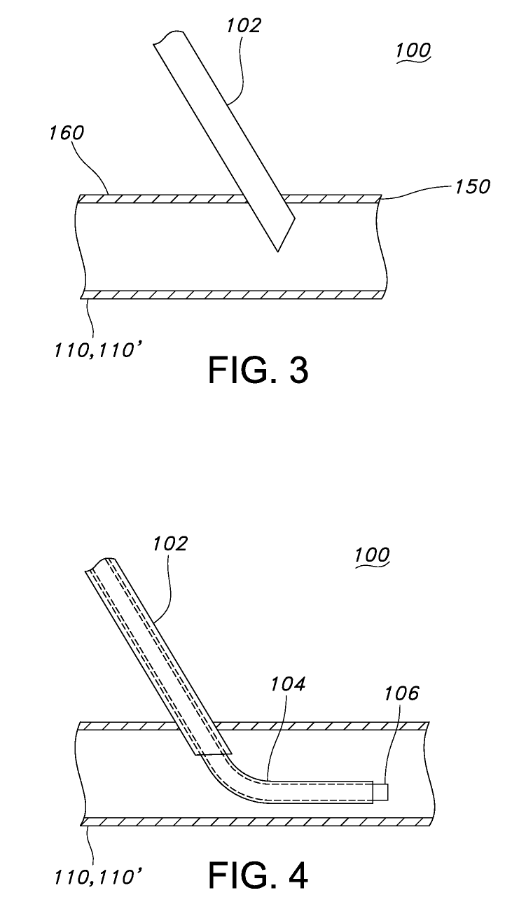

The following method illustrates a preferred embodiment for dialysis access according to the present invention. In order to obtain adequate blood flow from portions of the circulatory system near the surface, the surgeon/practitioner takes the surgical procedures, as illustrated from FIGS. 3 to 8 and as described below.

FIGS. 3 to 8, depict methods of using the dialysis needle system 100 of the present invention, which may comprises a needle 102 with an expandable sheath 104 surrounding a guide wire 106. The sheath 104 circumferentially surrounds the guide wire 106. The sheath 104 is deployed after insertion of the needle system and positioned within the vein or a dialysis fistula. The proximate end of the sheath 104 and the proximal end of the guide wire 106 are separated or otherwise configured so that each of the proximate ends can be attached to hoses attached to a hemodialyzer (not shown).

First, as can be seen in FIG. 3, a native 110′ or synthetic 110 vessel is punctured with the hollow needle 102.

Next, FIG. 4 shows that the expandable sheath 104 of the present invention is inserted through the needle 102 over a guide wire 106. Alternatively, the guide wire 106 can be used first, and the expandable sheath 104 introduced over the guide wire 106.

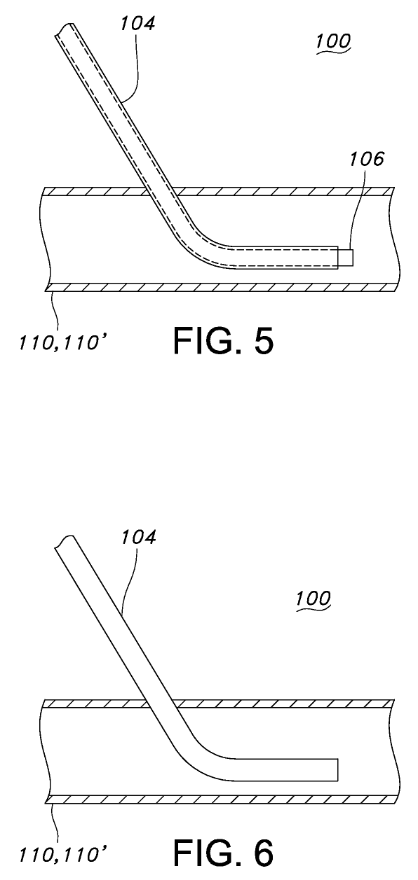

FIG. 5 shows the needle 102 being, withdrawn from the vessel 110, 110′. Alternatively, the needle 102 may be withdrawn prior to introducing the expandable sheath 104 over the guide wire 106.

FIG. 6 illustrates the next step where the guide were 106 is withdrawn. This step and the previous step may also, if desired, be performed in the opposite order.

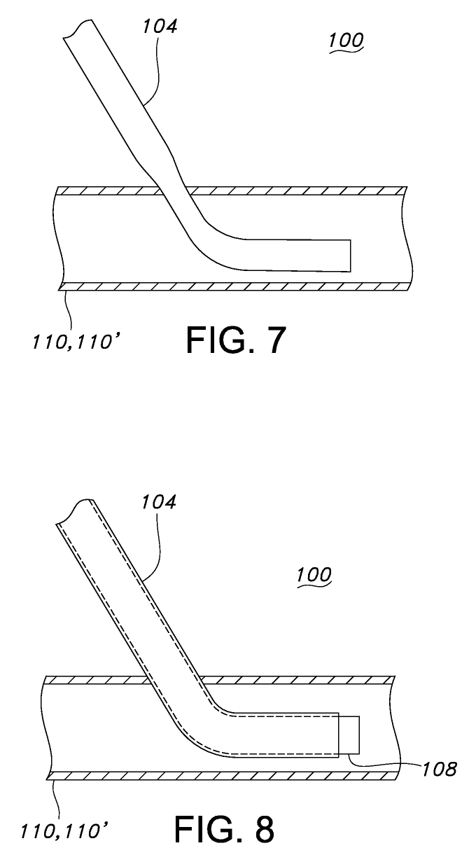

FIG. 7 depicts the expandable sheath 104 being expanded within the vessel 110, 110′. In some situations, this expandable sheath 104 may be used for passage of blood into or out of the vessel 110′ or stent-graft 110 disposed thereat, there along or there within the bodily vessel 110′. As shown in FIG. 7, the sheath 104 placed in a patient's dialysis conduit or vessel 110, 110′. The sheath 104 naturally expands by the pressure of the flowing blood and causes flow in dialysis sheath 104. Blood flows from one lumen via a sheath 104, through tubing to a hemodialyzer (not shown), through tubing, and then into another lumen via a sheath 104.

As depicted in FIG. 8, a supporting catheter 108 may optionally be introduced through the expandable sheath 104. In some situations, the internal supporting catheter can be used for passage of blood into or out of the vessel 110′ or stent-graft 110, or for passage of another intravascular device.

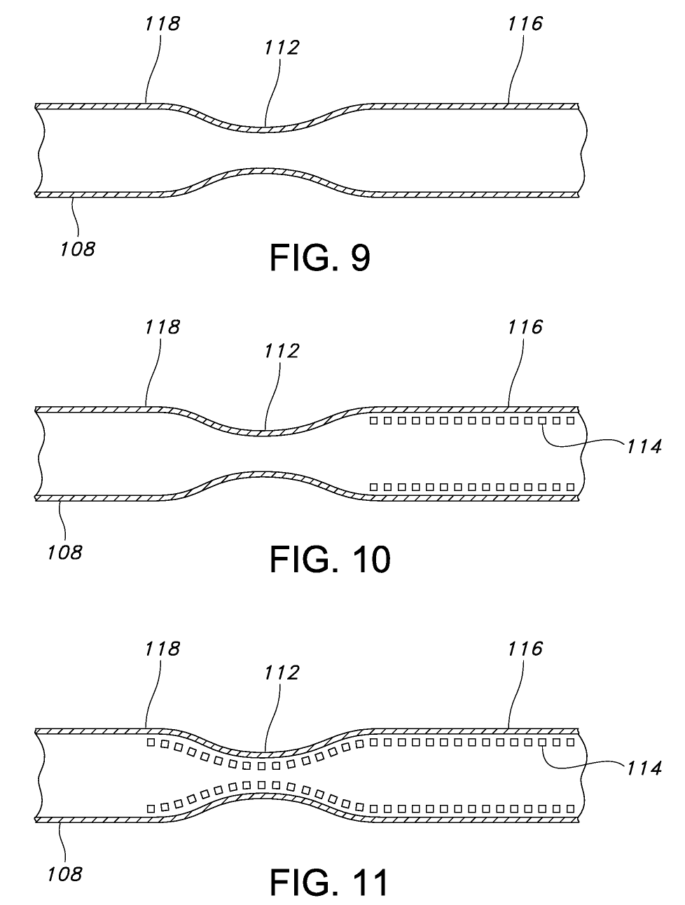

FIG. 9 illustrates schematically the internal supporting catheter 108 with a narrowed portion 112. The internal supporting catheter 108 can be a simple tubular catheter, or it can be configured to have a relatively short narrowed portion 112 to be located where the expandable sheath is passing into the vessel 110′ or graft 110, possibly including the tract through the tissue is well. This relatively short narrowed portion 112 may add some restriction to flow, but much less than if the entire length of the fluid path were narrowed, and provides reduced trauma, and tends to reduce pain and post-removal bleeding.

Various alternative configurations may be included with the internal supporting catheter 108. FIG. 10 illustrates a support element 114 along a portion 116 of the internal supporting catheter 108. The support element 114 may be a polymer layer, polymer fibers or bead, metal wire or mesh or other support.

Alternatively, the support element 114 may extend throughout the length of the internal supporting catheter 108, including portions 116 and 118 adjacently juxtaposed to the narrow portion 112, as can be seen in FIG. 11. The support element may utilize intravascular stent technology. For example, a self-expanding metal structure could be used, which would help to maintain the size and integrity of the passage. Particularly advantageous is an elastic structure which is formed to take the narrowed shape, as illustrated in the FIG. 11. Stainless steel, nickel-titanium alloys, and other metal and polymers, including shape-memory, heat-treated, spring, or other materials can be utilized for the support element, or the support function can be incorporated into the internal support catheter itself.

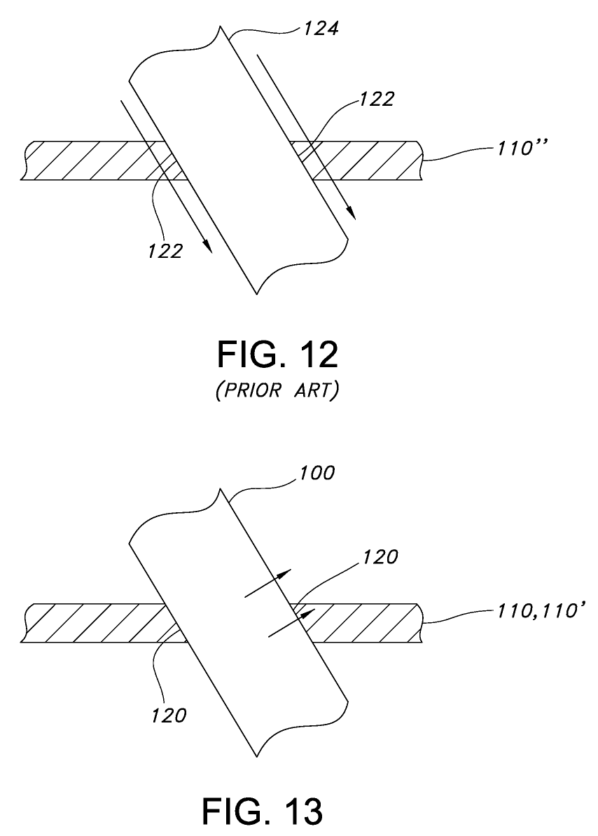

As depicted in FIGS. 12 and 13, the present invention provides reduced trauma compared to standard devices and methods. The initial puncture 120 with the present invention is a smaller diameter puncture, followed by primarily radial force expansion of the puncture tract. This is in contrast with the standard approach which utilizes a larger diameter puncture 122, which cutting and greater shearing forces by the larger prior art device or needle 124. The distinction is illustrated by the conventional puncture 122 through one wall of a vascular graft 110″ in the FIG. 12. In contrast, FIG. 13 illustrates a smaller puncture tract 120 being expanded according to the present invention.

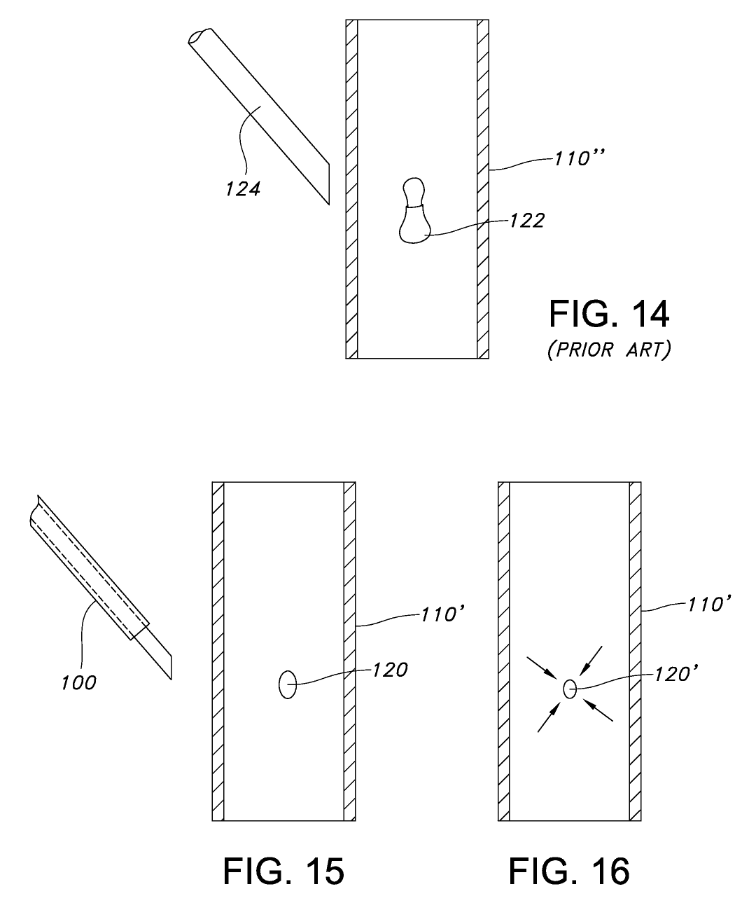

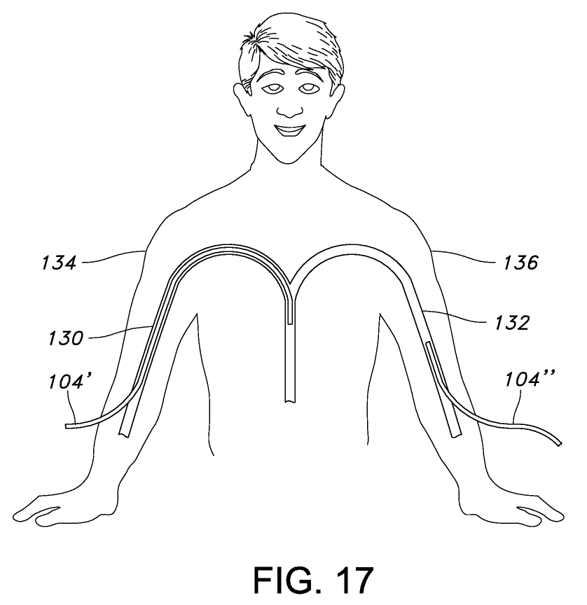

FIG. 14 depicts the puncture 122 of the prior art device 124 in a conventional ePTFE graft 110″. In comparison to the punctures tracts 120 of the present invention as depicted in FIG. 15, the prior puncture 122 is substantially larger. Moreover, as depicted in FIG. 16, the puncture 120′ reduces in size when the graft 110′ has some elastomeric properties, as contrasted to the non-elastomeric properties of ePTFE graft 110″ of the prior art.

The present invention may be utilized for venous access for chemotherapy or other diagnostic or therapeutic procedures as well as for dialysis access. One particularly advantageous application is veno-venous dialysis, which is not practical using existing devices and method.

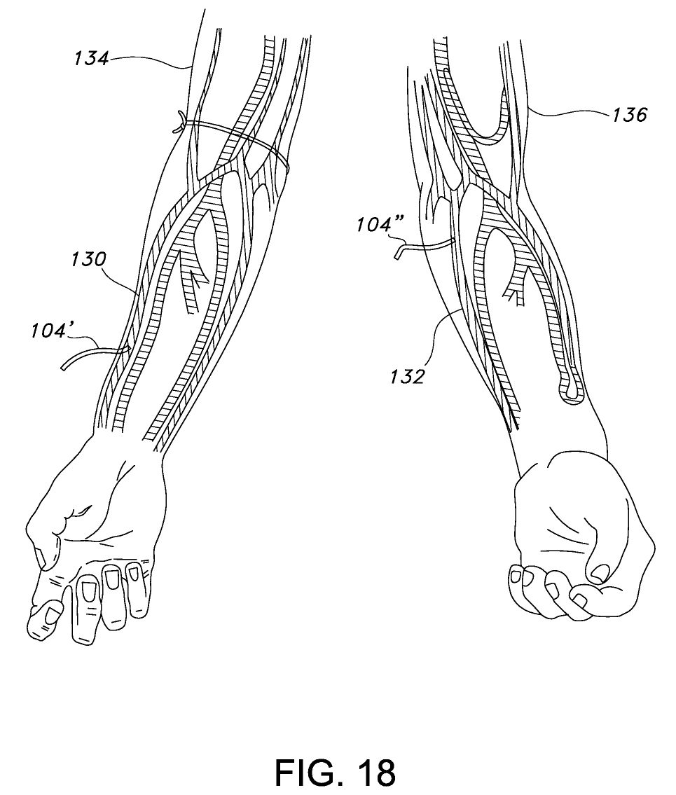

As illustrated in FIG. 17, expandable sheath 104′, 104″ of the present invention may be disposed into veins 130 and 132 of arms 134, 136, respectively. Sheath 104′ may be used to deliver blood, for example venous blood, to a dialyzer (not shown). Sheath 104″ may be used to deliver the blood from the dialyzer (not shown) to the vein 132. As depicted in FIG. 17, such veno-venous dialysis provides less veno-venous dialysis provides less risk of bleeding complications, faster homeostasis after cannula removal, and a voice the necessity of a permanent arteriovenous shunt. The present invention makes veno-venous dialysis possible by providing a less dramatic puncture for a large caliber catheter for center of venous access. A short vascular graft or intravascular stent-graft may be utilized to facilitate repeated punctures. A venous interposition graft, or venous stent-graft (which can be placed percutaneously), or multiple graft or stent-graft devices, can be utilized. Alternatively, a more conventional arteriovenous shunt may be created by using a fistula, graft, or stent-graft, and gaining the benefits of reduced puncture trauma, but not the benefits of venovenous access.

As depicted in FIG. 18, a human arm including arteries and veins is illustrated for carrying blood from the heart to the capillaries extending throughout the arm and the hand, and a larger number of veins for returning the blood to the heart. Although the arterial blood flow is at a considerably higher pressure and velocity than the venous blood flow, and the arteries are located in deeper parts of the arm than the veins.

The invention also includes methods of fabrication of the expendable dialysis sheath 104. One illustrative method includes the steps of extruding a thin walled tube, placing the tube over a mandrel, folding or wrapping the tube around the mandrel utilizing heat and pressure, and removing the tube from the mandrel. Another illustrative method includes the steps of extruding a thin walled polymeric tube, flattening the tube, wrapping the tube around a mandrel, and removing the tube from the mandrel. Other methods include the step of temporarily tacking the thin walled tube at one or more locations to temporarily retain it in the folded or wrapped configuration, utilizing heat, pressure, solvent, choose one or other energy source, ultrasonic welding, or adhesive material.

A variety of vascular grafts 110 and stent-grafts 110 can be utilized with the present invention. These include conventional ePTFE grafts, PET grafts, silicone rubber grafts, polyurethane grafts, other or other polymeric or elastomeric grafts, arterial grafts, venous grafts, or grafts which include biological material, coated or treated grafts, covered stents, stent grafts, and composite grafts. Alternatively, novel vascular grafts or stent-grafts can be utilized. Vascular grafts or stent-grafts such as those disclosed in U.S. Provisional Application No. 60/899,601, filed Feb. 5, 2007, corresponding patent publication with the applicant's docket number of 760-282P, titled, “Blood Access Apparatus and Method”, incorporated by reference, could be particularly well utilized with the present invention.

Useful vascular grafts 110 and stent-grafts 110 may also include micro-porous polymers. Examples of suitable micro-porous polymers are SIBS, polyurethane, PDMS, flouropolymer, proteins, PET, protein analogs, copolymers of at least one of these materials, or any other biologically stable and tissue-compatible materials as are known in the art. These polymers impart certain strength to the substructure and allow the graft wall to remain thin. Preferred micro-porous polymer materials are SIBS and PTFE. Thin-layer SIBS, in particular, has a proven clinical safely record and can be used as a carrier for drugs that prevent the proliferation of smooth muscle cells.

Typical methods of forming the graft 110 of the invention utilizing these polymers include methods by spinning, weaving, winding, solvent-forming, thermal forming, chemical forming, deposition, and combinations, and include porous coatings, castings, moldings, felts, melds, roams, fibers, microparticles, agglomerations and combinations thereof. The preferred forming methods for the preferred polymer materials, in particular, are spinning, winding, and electrostatic spinning.

The graft 110 of FIG. 3 may include a vascular prosthesis 110 comprising micro-porous SIBS [poly(styrene-isobutylene-styrene)] formed by layering small fibers together in random. The shape of the fiber may also be changed via polymer composition, dispersion solids level, or a post-fiber forming process like a solvent spray and/or heated compression. Fiber packing density (pore size) for a given fiber diameter can also be controlled by changing the above mentioned processing conditions as well as others depending on the method of fiber formation. SIBS fibers may be formed by drawing from the melt polymer or from a solvent dispersion. The fibers may also be formed by using an electrostatic process, whereby the fibers are drawn to the target surface via an electrical potential (difference). Fiber size may range from about 200 micrometer down to about 0.1 micrometer in diameter. Preferably, fibers reduced to about 5 micrometer may be used.

In yet another aspect of the invention, the vascular prosthesis 110 can have an additional high-strength material which provides attributes which may include increased suture retention strength, resistance to aneurysm, resistance to delamination, resistance to kinking, and/or thin wall and high porosity to facilitate tissue ingrowth and healing.

Examples of such high-strength materials are nitinol, stainless steel, titanium, algiloy, elgiloy, carbon, cobalt chromium, other metals or alloys, PET, ePTFE, polyimide, surlytn, and other materials known in the art; the high-strength materials may be configured in the form of wires, meshes, screens, weaves, braids, windings, coatings, or a combination thereof and may be fabricated by methods such as drawing, winding, braiding, weaving, mechanical cutting, EDM machining, thermal forming, CVD (chemical vapor deposition), laser cutting, e-beam cutting, chemical forming, and other processes known in the art. Preferred high-strength materials are CVD nitinol and wound or braided nitinol.

The additional high strength material may comprise a thin walled NiTi (nitinol) mesh, which may be formed so that uniaxial or biaxial expansion occurs in the presence of a directed force. The mesh can be made in the form of a sheet or tube via laser cutting or chemical vapor deposition (CVD). The elastic expansion ratio and direction is directly a function of the formed pattern. Elastic expansion ratios as large as 10:1 are possible. The force needed to expand the mesh is also a direct function of the mesh design (i.e., mesh thickness, pattern shape).

Alternatively, a high strength material formulation method such as wire winding(s) or braid can be utilized. A preferred configuration includes winding(s) or braid of metal wire extending along the length of the prosthesis. The required strength and flexibility can be obtained by wires approximately 12 to 60 micrometers in diameter, leaving openings approximately 20 to 200 micrometers between wires, although dimensions outside these ranges may be chosen for particular applications such as for vascular access, intravascular placement, venous use, trauma, etc.

Another preferred configuration of such high strength material utilizes “yarn” or “cable” of multiple very fine wires, such as a bundle of 7 wires approximately 8 to 17 micrometers in diameter yielding a bundle approximately 25 to 50 micrometers in diameter. Bundles of 2 to 19 wires are preferred. Metal wires are preferred, but other high strength fiber materials such as polymer or ceramic can be used, or a combination of materials.

In a further aspect of the invention, a vascular prosthesis 110 may utilize a leakage-reducing polymer in combination with a resorbable filler material, in a thin-walled structure. The bioresorbable material can provide initial structure, leakage resistance, or strength properties in a smaller-pore-size configuration, but which at least partially resorbs, degrades, dissolves, or otherwise transforms into a larger-pore size configuration to facilitate tissue ingrowth; optionally, the resorption of the bioresorabale material can facilitate elution of the biologically active material as well.

Examples of resorbable materials include gelatin, alginate, PGA, PLLA, collagen, fibrin and other proteins. Examples of leakage-reducing materials include ePTFE, hydrophobic coatings, and bioresorbable layers.

In yet another aspect of the present invention, a vascular prosthesis 110 comprises a thin multi-component tube, with micro-porous polymer, and also includes biologically active material which elutes, activates, or releases to modify the tissue response to the prosthesis. In essence, a flexible drug elution capability is provided so that growth factors, thrombosis inhibitors, platelet inhibitors, inflammatory inhibitors, cellular proliferation or migration modifying agents, or other agents can be included. Surface adsorption of these agents, binding agents, proteins or ligands, cells, or cellular precursors can also be accomplished due to the unique characteristics of the present invention.

In still another aspect of the present invention, the vascular prosthesis 110 can have a biologically active layer which reduces leakage of blood or serum.

In an additional aspect of the invention, the vascular prosthesis 110 can have a biologically active layer which reduces luminal thrombus formation from that which would otherwise occur.

Examples of suitable biologically active materials include antimicrobial agents, growth factors, hormones, anti-thrombosis materials, stenosis inhibitors, antibiotic agents, anti-tumor agents, antiproliferative agents, angiogenesis modulating agents, anti-mitotic agents, inflammation modulating agents, cell cycle regulating agents, genetic agents, and homologs, analogs, derivatives, fragments, compounts, and combinations thereof.

Preferred biologically active materials include agents which reduce tissue migration, proliferation, and hyperplasia which include but are not limited to paclitaxel, taxotere, raparnycin, sirolimus, and everolimus.

Other materials such as elastin, acellular matrix proteins, decellularized small intestinal submucosa (SIS), and protein analogs, and certain polymers such as PTFE can perform multiple functions such as providing micro-porous material, leakage-reducing function, bioresorption, and/or facilitation of elution of biologically active material.

In another aspect of the invention, biologically active material is located in different amounts or concentrations in different regions of the prosthesis to modify the tissue response differently in particular regions of the prosthesis.

In alternative configurations, agents call be included in selected portions of the prosthesis or the entire prosthesis. Further, the prosthesis can be configured to retain the agent(s), or release them over a short or long duration depending on the particular effects desired.

For example, anticoagulant or antiplatelet agents may be applied selectively to the luminal surface of the entire prosthesis, agents the stimulate endothelial proliferation and migration may be applied selectively to the subluminal portion away from the ends of the prosthesis, and cellular proliferation inhibitor may be applied selectively to one or both ends of the prosthesis, or a combination can be applied, with similar or varying duration of activity or elution rates. Regardless of the choice of any agents, the porous structure should allow tissue ingrowth through the wall of the prosthesis along the entire length of the prosthesis. The porous structure can allow tissue ingrowth through every portion of the wall or selected intermittent regions of the prosthesis can allow tissue ingrowth as long as the intermittent regions are present along the entire length of the prosthesis and are not spaced too far apart.

In a preferred exemplary embodiment, the vascular prosthesis comprises spun micro-porous polymer, anti-hyperplasia agent, and high-strength material of metal. A particular example of this preferred embodiment is fabricated of micro-porous SIBS polymer, paclitaxel, and nitinol.

Referring to the general configuration of FIG. 3, several exemplary embodiments of the invention shall be disclosed. It is noted that although the general configuration of the graft, stent-graft or vascular prosthesis 110 of FIG. 3 is cylindrical, a differing diameter width, or a flare at one end, or any other variation is envisioned by this invention.

The vascular prosthesis 110 as shown generally in FIG. 3 is further characterized by a proximal end 150 and a distal end 160. The wall structure of the vascular prosthesis 110 of the present invention may include a luminal ePTFE layer with a conformal coating of SIBS, ELS spun SIBS, a wire braid, and ELS spun SIBS as an exterior layer. Such preferred configuration of the present invention is particularly adapted for small-diameter applications (such as less than 5 mm in diameter). The details of each specific layer is as follows:

A luminal layer of very thin (20 to 100 micrometers, preferably about 25 micrometers) large-pore (40 to 150 micrometer intemodal distance, preferably about 60 micrometers) ePTFE has a thin (less than 1 micrometer) conformal coating of SIBS which does not modify the fibroporous structure of the ePTFE significantly.

A first interconnected microfibrous mat of SIBS may be fabricated on the external surface of the coated ePTPE; the mat has small fibers (less than about 50 micrometers, preferably about 25 micrometers) and large pores (greater than about 40 micrometers, preferably about 60 micrometers) such as can be created using spinning technology such as electrostatic spinning (preferably about 100 micrometers overall thickness).

The wire braid or windings may be applied on the external surface of the SIBS mat and may slightly compress the mat at the locations of the wires. The braid desirably includes fine wires or bundles (less than about 50 micrometers, preferably about 25 micrometers) and spacing to allow tissue ingrowth yet provide strength and support (50 to 300 micrometers, preferably about 100 micrometers).

A second interconnected microfibrous mat of SIBS may be fabricated on the external surface of the coated wire braid, similar to the first mat (but preferably about 50 micrometers overall thickness). A final solvent or dilute SIBS solution can be applied to help bond the various layers of SIBS into a stable structure.

Preferably, any of the SIBS components may include one or more agents such as those described above. A hemostatic coating or filler material is preferably used to reduce leakage of blood in the period prior to tissue ingrowth; suitable materials include protein-based materials such as collagen, elastin, fibrin, albumen, gelatin, submucosa, extracellular matrix, decellularized tissue materials, plant-based degradable polymers, synthetic analogs of these protein based materials, lipid-based materials, gels, PEG, PGA, PLLA, and other polymers and copolymers. Certain of these materials may be less suitable due to acidic degradation products, pro-inflammatory effect, stiffness or brittleness, or rate of degradation; preferred materials include protein-based materials, glycols, lipid-based materials, and their analogs.

Alternatively, the vascular prosthesis 110 may further comprise a hemostatic filler material. Such a prosthesis 100 may include a luminal ePTFE layer with a conformal coating of alginate, ELS spun alginate, wire braid, and ELS spun alginate as the exterior layer. Alternatively, the vascular prosthesis 110 may include a graft lumen surface on the luminal layer. An interconnected microfibrous mat of pSIBS may be provided on top of the graft lumen surface. Then, a metal braid may be applied on the external surface of the pSIBS mat. A layer of SIS impregnated SIBS is fabricated on the external surface of the metal braid. A final solvent or dilute SIBS can be applied to help bond the layers into a stable vascular prosthesis.

Still further alternate designs may include several possible combinations, which may include, but not necessarily exclude any other variations mentioned above. The alternate designs ray include ePTFE, alginate, PLA, PLLA, PGA, SIS, elastin, PEG, or gelatin as the first, second or third layer; flat or round filament braid or helical wrap of elgiloy, stainless steel, tantalum, platinum, nitinol, cobalt chromium, or other metal, or PET or PTFE fibers as the reinforcement layer; sirolimus, growth inhibitors, inflammatory modulators, or locally toxic materials as the biologically active material.

it has been found that the average pore distance, as measured along the axis of expansion, should preferably fall within a relatively narrow range of values between approximately 1 and 80 microns. As will be appreciated by those skilled in the art, the term “average” when used in conjunction with spacing distance and pore size cannot be used or interpreted with statistical precision; rather, the term is intended to connote a nominal or typical dimension derived from a broad sample. By way of example, where the average spacing distance is said to be 30 microns, it would be expected that some of the pores would be separated by only a few microns while others might be separated by 90 or 100 microns. In the ideal vascular prosthesis, each pore would have a perfect elongated football shape and would be separated from its neighbors by uniformly distributed fibrils 10 of equal lengths. Unfortunately, such perfection is rarely, if ever, achieved in a microscopic environment.

Where the average spacing distance is less than the major dimension of a typical red cell, or approximately 6 microns, inadequate cellular ingrowth has been observed. In such cases, the pore/fibril superstructure is so tightly packed as to preclude either the establishment or continued nutrition of a viable neointima.

Associated with (very large spacing distances is a loss of tensile strength and overall structural integrity. The vascular prosthesis becomes progressively more pliable and progressively more difficult to handle during surgery. Excessively expanded vascular prostheses will be subject to deformation and leakage at the suture line. Furthermore, excessive cellular ingrowth has been observed in vascular prostheses having an average spacing in excess of approximately 80 microns. Where the inside diameter of the vascular prosthesis is critically small, excessive cellular penetration of this type can lead to the formation of a pseudointima or an unacceptable thickening of the neointima with an accompanying occlusion of the lumen.

As the average spacing distance is extended beyond, for example, 150 to 200 microns, the vascular prosthesis superstructure becomes progressively more permeable to blood flow and is characterized by substantial interstial clotting and progressively decreasing and non-uniform cellular ingrowth. Ultimately, were it possible to reproduce spacing distances comparable in size to the interstial voids of the size characterizing woven vascular prostheses, then virtually all transmural growth would be inhibited, and the support of a true neointima would be impossible.

The present invention also includes various methods of manufacturing the sheath and the catheter therein. Polytetrafluoroethylene is extruded to form tubing having an average inside diameter of approximately 4 millimeters and an average wall thickness of approximately 0.5 millimeters. Unsintered tubing of this type, identified by the manufacturer's No. S16882-7, may be obtained from W. S. Shamban Company (71 Mitchell Road, Newberry Park, Calif. 91320). The unsintered extrudate, which is quite fragile, is carefully cut with a razor blade into lengths of, for example, 7.3 centimeters. Small aluminum plugs of virtually any configuration are inserted into the end of the tubing and secured thereto by tightly wrapped stainless steel wire. A relatively short end segment is thus confined between the inserted plug and the stainless steel wire. These plugs provide points for handling and attachment during the subsequent heating, elongation and sintering steps.

The tubing and plug assembly is placed in a uniformly heated oven for approximately one hour at 275° C. Thereafter, the assembly is removed from the oven, and the plugs are grasped and stretched apart manually to obtain a tubular length of 23 centimeters. The time required for removal and elongation should be made as short as possible to reduce the effects of cooling. Elongation should be carried out at a moderate, uniform rate and the plugs should be moved apart along a common axis of expansion to assure uniform force distribution. Typically, this manual operation has required less than ten seconds and has yielded good results.

The elongated assembly is then secured against contraction ba restraining the plugs at the desired separation. This may be achieved in any number of obvious ways, as for example, by using plugs with enlarged ends which are placed in a fixture having U-shaped slots separated by the desired distance of 23 centimeters.

While still restrained, the elongated assembly is returned to the oven for approximately forty-five seconds at 400° C., during which time the pore/fibril superstructure is sintered and becomes fixed. The elongated vascular prostheses are then cut to the desired lengths and after sterilization are ready for implantation.

In large commercial applications, expansion is achieved mechanically in the oven itself at closely controlled rates and is immediately followed by sintering.

Fabrication of tapered vascular prostheses such as those used for peripheral artery replacement involves the additional step of reshaping a sintered tube of desired length and diameter over a tapered stainless steel mandrel which has been heated to approximately 300° C. After the entire assembly is allowed to cool, the slightly re-expanded vascular prosthesis retains the shape of the mandrel and may be removed for use without further heat treatment.

Various other configurations such additional layers of the materials or structures cited above may also be produced. Accordingly, it is intended by the appended claims to cover all such modifications of the invention which fall within the true spirit and scope of the invention.

It will be apparent to those skilled in the art that the disclosed method of treatment may be modified in numerous ways and may assume many embodiments and configurations other than those specifically set forth and described above. For example, the basic structure may be enhanced with biologically active material to reduce infections, reduce inflammation, reduce thrombosis, or encourage healing, or encourage endothelialization of the vascular graft to improve the treatment.

The following embodiments or aspects of the invention may be combined in any fashion and combination and be within the scope of the present invention, as follows:

### EMBODIMENT 1

A dialysis needle system comprising: a blood feeding needle component configured to introduce blood at a position of a first peripheral vein and a blood withdrawal needle component configured to withdraw blood at another position from a second peripheral vein located, wherein said first position is located away from said second position.

### EMBODIMENT 2

A dialysis needle system according to embodiment 1, wherein said withdrawal needle component further comprises: a guide wire having a central axis, a proximal end and a distal end; an expanding sheath configured circumferentially around said guide wire to form an annular lumen between a distal blood withdrawal position and a proximal extracorporeal position; and a needle disposed around said expandable sheath.

### EMBODIMENT 3

A dialysis needle system according to embodiment 1, wherein said position and said another position are made with a small trauma minimizing puncture hole.

### EMBODIMENT 4

A dialysis needle system according to embodiment 2, wherein the feature of said expanding sheath is provided by a folded sheath.

### EMBODIMENT 5

A dialysis needle system according to embodiment 1, wherein said position and said another position are provided at a native vessel.

### EMBODIMENT 6

A dialysis needle system according to embodiment 1, wherein said position and said another position are provided at a prosthetic graft

### EMBODIMENT 7

A dialysis needle system according to embodiment 1, wherein said position and said another position are provided at an intravascular graft.

### EMBODIMENT 8

A dialysis needle system according to embodiment 2, wherein said expandable sheath stays small enough to travel from a peripheral vein to a central venous vessel without substantially impeding the vein flow.

### EMBODIMENT 9

A dialysis needle system according to embodiment 2, wherein said sheath includes substantially larger diameter along a substantial portion of the cannula to reduce resistance to flow of blood or other fluids.

### EMBODIMENT 10

A dialysis needle system according to embodiment 1, wherein said needle has an outer diameter less than 14 gauges.

### EMBODIMENT 11

A dialysis needle system according to embodiment 2, wherein said needle is configured to provide for reduced shear forces on access puncture, and provides enhanced radial expansion of the puncture.

### EMBODIMENT 12

A dialysis cannula comprising: a guidewire having a central axis, a proximal end and a distal end; a sheath configured to an enlarging feature that enlarges the radial circumference around said guidewire to form an annular lumen therebetween and stay small enough to travel through a vein without substantially impeding the vein flow, the sheath having a proximal end and a distal end; and a needle disposed around said expandable sheath.

### EMBODIMENT 13

A dialysis needle system according to embodiment 12, further including a supporting catheter.

### EMBODIMENT 14

A dialysis needle system according to embodiment 12, wherein said sheath is configured to expand at the skin level.

### EMBODIMENT 15

The needle system of embodiment 12, wherein the sheath can be moved slidably over the needle.

### EMBODIMENT 16

The needle system of embodiment 12, wherein the outer sheath slides over the inner sheath in only the longitudinal direction.

### EMBODIMENT 17

A single access dialysis needle system comprising: a cannula having a proximal end and a distal end, and a lumen extending therethrough, an inner sheath arranged circumferentially around the cannula to form an annular lumen between the cannula and the inner sheath, the inner sheath having a proximal end and a distal end, an outer sheath arranged circumferentially around the inner sheath and having a proximal end and a distal end, and a barrier capable of being deployed to extend substantially radially from the outer sheath to block or substantially obstruct fluid flow, wherein the proximal end of the cannula and the annular lumen are adapted for connection to a blood hemodialyzer, and the inner sheath and the outer sheath each have one or more lateral openings.

### EMBODIMENT 18

The needle system of embodiment 17, wherein the lateral openings are positioned so that the outer sheath can be slid over the inner sheath to cause at least two lateral openings to align.

### EMBODIMENT 19

The needle system of embodiment 4, wherein the barrier blocks or partially obstructs blood in a blood vessel.

### EMBODIMENT 20

A single access dialysis needle system comprising: a cannula having a proximal end and a distal end, and a lumen extending therethrough, an inner sheath arranged circumferentially around the cannula to form an annular lumen between the cannula and the inner sheath, the inner sheath having a proximal end and a distal end, and a guide wire capable of being deployed to extend substantially radially from the sheath to guide sheath, wherein the proximal end of the cannula and the annular lumen are adapted for connection to a blood hemodialyzer, and, wherein the distal end of the cannula comprises a dilating member arranged circumferentially around said distal end.

### EMBODIMENT 21

The needle system of embodiment 20, wherein the distal end of the inner sheath is affixed to or integral with the dilating member.

### EMBODIMENT 22

The needle system of embodiment 20, wherein the distal end of the outer sheath is affixed to or integral with the dilating member.

### EMBODIMENT 23

The needle system of embodiment 20, wherein hemostasis valve is circumferentially arranged around the proximal end of the cannula, the proximal end of the inner sheath, or both.

### EMBODIMENT 24

The needle system of embodiment 20, wherein the hemostasis valve is in fluid communication with a port.

### EMBODIMENT 25

The needle system of embodiment 20, wherein the hemostasis valve can be moved in a longitudinal direction relative to the needle, the inner sheath, or both.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

What is claimed is:

1. A dialysis needle system comprising:
a blood feeding needle component configured to introduce blood at a position of a first peripheral vein and a blood withdrawal needle component configured to withdraw blood at another position from a second peripheral vein located, wherein said first position is located away from said second position.

2. A dialysis needle system according to claim 1, wherein said withdrawal needle component further comprises:
a guide wire having a central axis, a proximal end and a distal end;
an expanding sheath configured circumferentially around said guide wire to form an annular lumen between a distal blood withdrawal position and a proximal extracorporeal position; and
a needle disposed around said expandable sheath.

3. A dialysis needle system according to claim 1, wherein said position and said another position are made with a small trauma minimizing puncture hole.

4. A dialysis needle system according to claim 2, wherein the feature of said expanding sheath is provided by a folded sheath.

5. A dialysis needle system according to claim 1, wherein said position and said another position are provided at a native vessel.

6. A dialysis needle system according to claim 1, wherein said position and said another position are provided at a prosthetic graft

7. A dialysis needle system according to claim 1, wherein said position and said another position are provided at an intravascular graft.

8. A dialysis needle system according to claim 2, wherein said expandable sheath stays small enough to travel from a peripheral vein to a central venous vessel without substantially impeding the vein flow.

9. A dialysis needle system according to claim 2, wherein said sheath includes substantially larger diameter along a substantial portion of the cannula to reduce resistance to flow of blood or other fluids.

10. A dialysis needle system according to claim 1, wherein said needle has an outer diameter less than 14 gauges.

11. A dialysis needle system according to claim 2, wherein said needle is configured to provide for reduced shear forces on access puncture, and provides enhanced radial expansion of the puncture.

12. A dialysis cannula comprising:
a guidewire having, a central axis, a proximal end and a distal end;
a sheath configured to an enlarging feature that enlarges the radial circumference around said guidewire to form an annular lumen therebetween and stay small enough to travel through a vein without substantially impeding the vein flow, the sheath having a proximal end and a distal end; and
a needle disposed around said expandable sheath.

13. A dialysis needle system according to claim 12, further including a supporting catheter.

14. A dialysis needle system according to claim 12, wherein said sheath is configured to expand at the skin level.

15. The needle system of claim 12, wherein the sheath can be moved slidably over the needle.

16. The needle system of claim 12, wherein the outer sheath slides over the inner sheath in only the longitudinal direction.

17. A single access dialysis needle system comprising:
a cannula having a proximal end and a distal end, and a lumen extending therethrough, an inner sheath arranged circumferentially around the cannula to form an annular lumen between the cannula and the inner sheath, the inner sheath having a proximal end and a distal end, an outer sheath arranged circumferentially around the inner sheath and having a proximal end and a distal end, and a barrier capable of being deployed to extend substantially radially from the outer sheath to block or substantially obstruct fluid flow, wherein the proximal end of the cannula and the annular lumen are adapted for connection to a blood hemodialyzer, and the inner sheath and the outer sheath each have one or more lateral openings.

18. The needle system of claim 17, wherein the lateral openings are positioned so that the outer sheath can be slid over the inner sheath to cause at least two lateral openings to align.

19. The needle system of claim 4, wherein the barrier blocks or partially obstructs blood in a blood vessel.

20. A single access dialysis needle system comprising: a cannula having a proximal end and a distal end, and a lumen extending therethrough, an inner sheath arranged circumferentially around the cannula to form an annular lumen between the cannula and the inner sheath, the inner sheath having a proximal end and a distal end, and a guide wire capable of being deployed to extend substantially radially from the sheath to guide sheath, wherein the proximal end of the cannula and the annular lumen are adapted for connection to a blood hemodialyzer, and, wherein the distal end of the cannula comprises a dilating member arranged circumferentially around said distal end.

21. The needle system of claim 20, wherein the distal end of the inner sheath is affixed to or integral with the dilating member.

22. The needle system of claim 20, wherein the distal end of the outer sheath is affixed to or integral with the dilating member.

23. The needle system of claim 20, wherein hemostasis valve is circumferentially arranged around the proximal end of the cannula, the proximal end of the inner sheath, or both.

24. The needle system of claim 20, wherein the hemostasis valve is in fluid communication with a port.

25. The needle system of claim 20, wherein the hemostasis valve can be moved in a longitudinal direction relative to the needle, the inner sheath, or both.
